(19) United States
(12) Patent Application Publication
(10) Pub. No.: US 2006/0266649 A1
(43) Pub. Date: Nov. 30, 2006

(54) Electrophoretic member, electrophoretic device, electrophoretic method and sample dispensing probe

(75) Inventors: Shin Nakamura, Moriyama-shi (JP); Rintaro Yamamoto, Kyoto-shi (JP); Toru Kaji, Kyoto-shi (JP)

Correspondence Address: KANESAKA BERNER AND PARTNERS LLP, SUITE 300, 1700 DIAGONAL RD, ALEXANDRIA, VA, 22314-2848, US

(73) Assignee: Electrophoretic Member

(21) Appl. No.: 10/571,893

(22) PCT Filed: Sep. 21, 2004
(86) PCT No.: PCT/JP04/14146

§ 371 Date: Mar. 14, 2006

(30) Foreign Application Priority Data
Sep. 22, 2003 (JP) 2003-330614

(51) Int. Cl. C07K 1/26; G01N 27/00

(52) U.S. Cl. 204/453; 204/601

(57) ABSTRACT

An electrophoretic member is structured such that a plate member as a base plate is provided with a separating flow path in a shape of a groove, and another plate member is provided with through-holes as small reservoirs at positions corresponding to both ends of the flow path. Both plate members are bonded together, so that the flow path is disposed inside thereof and both ends of the flow path communicate with the reservoirs to constitute an integrated base plate. Larger reservoirs having a size greater than that of the small reservoirs are connected at the positions of the small reservoirs on the plate member. Thus, a sample injection quantity can be minimized.

### BACKGROUND OF THE INVENTION AND RELATED ART STATEMENT

The present invention relates to an electrophoretic member, an electrophoretic device using the electrophoretic member, an electrophoretic method using the electrophoretic member, and a sample dispensing probe for analyzing an extremely small quantity of protein, nucleic acid, chemicals, or the like such as DNA sequence at a high speed with a high resolution in the fields of biochemistry, molecular biology and clinic.

When an extremely small quantity of protein, nucleic acid, or the like is analyzed, an electrophoretic device such as a capillary electrophoretic device has been conventionally used. Such a capillary electrophoretic device has a problem in a complicated handling. In view of the problem, an electrophoretic member so called a micro-fluid device having a channel in a base plate has been proposed to perform a high speed analysis and reduce a size of a device. An example of the micro-fluid device is shown in FIG. 12.

As shown in FIG. 12, in a micro-fluid device 1, a pair of plate members is bonded together to form a base plate, and loading channels 3 for introducing a sample and a separating channel 5 for electrophoretic migration crossing each other are formed in a joining surface of the base plate with a micro-machining technique. One of the plate members is provided with through-holes as an anode reservoir 7a, a cathode reservoir 7c, a sample reservoir 7s and a waste reservoir 7w at positions corresponding to ends of the channels 3 and 5. The micro-fluid device has the two channels crossing each other, and is thus called a cross-channel micro-chip.

When the micro-fluid device 1 performs an electrophoretic migration, prior to the analysis, a migration medium is pressurized and filled in the channels 3 and 5 and reservoirs 7a, 7c, 7s and 7w from one of the reservoirs, for example, the anode reservoir 7a with, for example, a syringe.

The conventional micro-fluid device performs the electrophoretic migration using the channels according to the cross injecting design shown in FIG. 12 (refer to Japanese Patent Publications (Kokai) No. 2002-131279, No. 2002-131280, No. 2002-310990, and No. 2003-166975). In the cross injecting method, a sample is injected as follows:

1) The sieving medium in the sample reservoir 7s is removed, and a sample is injected in the sample reservoir 7s.

2) The sample is uniformly and electrophoretically introduced into the loading channel 3, so that the sample migrates uniformly and does not migrate to a side of the separating channel 5 at a cross portion 9. At this time, the sample is, introduced while a voltage such as pinching is applied to plural positions. When a high viscosity gel is used for separating more than one base such as DNA sequence with a high resolution, the pinching operation is not necessary, and the voltage is applied just to the loading channel.

3) The voltage is switched to the side of the separating channel 5. At the same time, a voltage is applied to the loading channel 3, so that the sample is moved in a reserve direction from the cross portion 9 to thereby introduce the sample only in the cross portion 9 into the separating channel 5 to perform the electrophoretic separation. A nozzle sucks the sample, and the sample is discharged to a predetermined position of the analyzing device through the nozzle, so that the sample is injected to the electrophoretic device and other analyzing devices.

In the cross injecting method, the sample is injected into the sample reservoir 7s, and only an extremely small portion of the sample is introduced into the separating channel 5. Accordingly, it is necessary to inject a relatively large quantity of a sample, normally 5 to 20 μL (micro-liter), into the sample reservoir 7s. In the analysis such as DNA sequence, there may be a case in which only an extremely small amount of a sample is available. In the cross injecting method, it is difficult to analyze an extremely small amount of a sample, such as several tens of nL to a few μL. Also, when a sample is sucked with a nozzle and is discharged through the nozzle, it is difficult to inject a small quantity.

In view of the problems described above, an object of the invention is to provide an electrophoretic member, an electrophoretic device using the electrophoretic member, an electrophoretic method using the electrophoretic member, and a sample dispensing probe capable of handling an extremely small quantity of a sample.

Further objects and advantages of the invention will be apparent from the following description of the invention.

### SUMMARY OF THE INVENTION

To attain the objects described above, according to a first aspect of the present invention, an electrophoretic member is structured to improve efficiency of a sample so that an extremely small amount of the sample can be analyzed. The electrophoretic member includes at least one separating channel formed in a base plate and having a single channel so that a sample is electrophoretically separated along the channel when a voltage is applied between both ends thereof; a first reservoir disposed at both end portions of the separating channel and communicating with the separating channel for reserving liquid; and a second reservoir for injecting the sample provided inside the first reservoir disposed at one of the end portions of the separating channel at a sample injecting side.

The second reservoir may be formed in a small depression having a diameter smaller than that of the first reservoir. In that case, it is preferable that the second reservoir has an inner wall with hydrophilicity and the first reservoir has at least a bottom surface with hydrophobicity. The second reservoir may be provided in the bottom surface of the first reservoir. The second reservoir may be provided in the bottom surface of the first reservoir such that a peripheral surface of a portion communicating with the separating channel is processed to be hydrophilic and an outer portion thereof is processed to be hydrophobic.

Integrated electrodes may be provided in the first and second reservoirs in advance, respectively. The electrophoretic member may be formed of one base plate provided with one separating channel, or a common base plate provided with a plurality of separating channels.

According to a second aspect of the present invention, an electrophoretic device includes the electrophoretic member described above; a power supply device for applying a migration voltage between the both ends of the separating channel; a sample dispensing probe for dispensing a minute amount of a sample into the sample injecting second reservoir; and a detecting device disposed at an end portion of the separating channel opposite to the sample injecting side for detecting a sample component migrating along the separating channel.

According to a third aspect of the present invention, a sample dispensing probe can inject an extremely minute amount of a sample. The sample dispensing probe has a depression or a groove provided at a leading end thereof, so that an extremely minute amount of the sample is sucked and dispensed through capillary phenomenon and/or surface tension. The sample dispensing probe is applicable to the electrophoretic device of the invention as well as to an electrophoretic device of a cross injection system and other analyzing devices, in which an extremely minute quantity of a sample needs to be injected.

According to a fourth aspect of the present invention, in an electrophoretic method, the electrophoretic member of the invention is used, and the electrophoretic separation is sequentially performed with the following steps:

a step of filling the sieving medium into the separating channel;

a step of injecting the sample into the sample injecting second reservoir from the first reservoir on the sample injection side, and filling the migration buffer to the other first reservoir so that the sample is electrophoretically introduced into the channel;

a step of filling the migration buffer into the first reservoir on the sample injecting side while leaving the sample in the sample injecting second reservoir after the sample is injected; and

a step of applying a migration voltage between both ends of the separating channel for the electrophoretic separation.

In this case, even if the sample remains in the sample injecting second reservoir in the step of injecting the sample into the sample injecting second reservoir, the sample is not removed or cleaned. And, the migration buffer is filled to dilute the sample, and the electrophoretic separation is performed as it is. In the invention, after the sample is injected, even if the sample is not removed or cleaned, there is no influence on a migrating pattern. Accordingly, it is easy to operate the electrophoretic member.

In the first aspect of the invention, the electrophoretic member does not include a loading channel crossing the separating channel. The sample is injected into the sample injecting second reservoir, and is directly introduced into the separating channel. Since the sample is not introduced into a loading channel, it is possible to reduce a sample amount necessary for the analysis to an extremely minute quantity.

In the conventional cross injecting method, when a sample is introduced, the sample migrates and is separated even in the sample loading channel. Depending on a composition of the loading buffer, a stacking (condensation) phenomenon may occur in the loading channel, thereby causing a non-uniform sample distribution in the loading channel. Only a part of the sample introduced into the loading channel is introduced into the separating channel. Accordingly, the sample introduced into the separating channel may not have a composition same as that of the original sample. On the other hand, in the electrophoretic member of the invention, the stacking does not occur in the loading channel and the sample concentration distribution becomes uniform, so that the sample is introduced into the separating channel while maintaining the same concentration distribution, as compared with the conventional cross injecting method using the loading channel.

In the invention, the second reservoir may be formed in a depression having a diameter smaller than that of the first reservoir. It is preferable that the second reservoir has an inner wall with hydrophilicity and the first reservoir has at least a bottom surface with hydrophobicity. Accordingly, it is possible to agglomerate the sample selectively in the second reservoir, so that the injected sample can be effectively introduced into the separating channel. The peripheral surface of the portion communicating with the separating channel at the bottom surface of the first reservoir is processed to have hydrophilicity, and the outer side thereof is processed to have hydrophobicity. With this structure, it is easy to form the second reservoir in the bottom surface of the first reservoir. When the integrated electrode is disposed in the first reservoirs in advance, the electrode can contact the sample with an extremely minute amount.

In the third aspect of the present invention, the sample dispensing probe can suck and dispense an extremely minute amount of the sample through capillary phenomenon and/or surface tension. Accordingly, it is easy to inject a minute quantity of the sample as compared with a conventional method in which a nozzle sucks and discharges a sample.

### BRIEF DESCRIPTION OF THE DRAWINGS

FIGS. 1(A) and 1(B) are views showing an electrophoretic member according to an embodiment of the present invention, wherein FIG. 1(A) is a plan view thereof, and FIG. 1(B) is a sectional view taken along line X-X in FIG. 1(A);

FIGS. 2(A) and 2(B) are views showing an electrophoretic member according to another embodiment of the present invention, wherein FIG. 2(A) is a plan view thereof, and FIG. 2(B) is a sectional view taken along line Y-Y in FIG. 2(A);

FIG. 3 is an enlarged sectional view showing an electrophoretic member according to a further embodiment of the present invention;

FIG. 4 is a sectional view showing an electrophoretic member according to a still further embodiment of the present invention;

FIGS. 5(A) to 5(C) are views showing an electrophoretic member according to a still further embodiment of the present invention, wherein FIG. 5(A) is a plan view showing channels and first reservoirs; FIG. 5(B) is a partially enlarged view showing a second reservoir portion on a sample injection side; and FIG. 5(C) is a perspective view of the sample injecting side;

FIG. 6 is a perspective view showing an electrophoretic device according to the present invention;

FIGS. 7(A) and 7(B) are sectional views showing examples of a dispensing nozzle of a sample dispensing mechanism;

FIGS. 8(A) and 8(B) are schematic sectional views showing examples of the dispensing nozzles of the sample dispensing mechanisms together with transfer mechanisms;

FIGS. 9(A) and 9(B) are charts showing migrating patterns, wherein FIG. 9(A) is a result of Example 1, and FIG. 9(B) is a result of Comparative Example;

FIG. 10 is a graph showing a result of resolution compared between Example 1 and Comparative Example;

FIG. 11 is a graph showing a result of peak height compared between Example 1 and Comparative Example;

FIG. 12 is a plan view showing a conventional electrophoretic member;

FIGS. 13(A) to 13(H) are graphs showing migration patterns of Example 2, wherein FIG. 13(A) is a result of the 129th separation channel of the electrophoretic member from the left side, FIG. 13(B) is a result of the 130th separation channel of the electrophoretic member from the left side, FIG. 13(C) is a result of the 131st separation channel of the electrophoretic member from the left side, FIG. 13(D) is a result,of the 132nd separation channel of the electrophoretic member from the left side, FIG. 13(E) is a result of the 133rd separation channel of the electrophoretic member from the left side, FIG. 13(F) is a result of the 134th separation channel of the electrophoretic member from the left side, FIG. 13(G) is a result of the 135th separation channel of the electrophoretic member from the left side, and FIG. 13(H) is a result of the 136th separation channel of the electrophoretic member from the left side; and

FIG. 14 is a graph showing resolution of Example 2 in which the separation channel is the 129th from the left side.

### DETAILED DESCRIPTION OF PREFERRED EMBODIMENTS

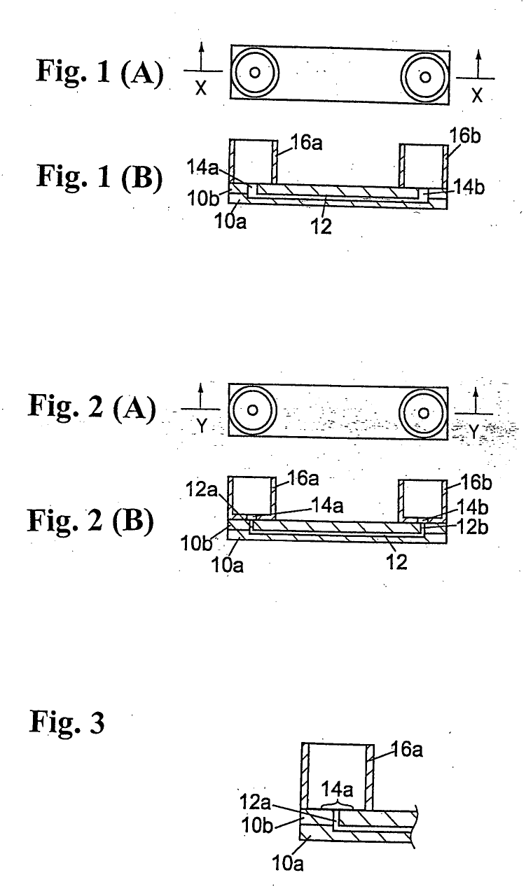

Hereunder, embodiments of the present invention will be explained with reference to the accompanying drawings. FIGS. 1(A) and 1(B) are views showing an electrophoretic member according to a first embodiment of the present invention.

A base plate is formed of a pair of plate members 10a and 10b connected to each other. A separating channel 12 is formed in one of the plate members 10a as a single groove. The channel 12 has a width of 100 nm to 1,000 μm, preferably 50 to 90 μm, and a depth of 100 nm to 1,000 μm, preferably 20 to 40 μm. Through-holes 14a and 14b are formed in the other of the plate members 10b as second reservoirs at positions corresponding to both ends of the channel 12. The second reservoir has a diameter of 10 μm to 3 mm, preferably 50 μm to 2 mm, to have a size suitable for filling a sample of several tens of nL to a few μL. The plate members 10a and 10b are bonded together to form an integrated base plate, in which the channel 12 is disposed inside the integrated base plate and both ends of the channel 12 are connected to the second reservoirs 14a and 14b.

First reservoirs 16a and 16b are attached to the plate member 10b at positions corresponding to the second reservoirs 14a and 14b, respectively. Each of the first reservoirs is formed of a cylindrical member having a diameter larger than that of the second reservoirs 14a and 14b, and is connected to the plate member 10b. The first reservoirs 16a and 16b are positioned so that each of the second reservoirs 14a and 14b is positioned at a central portion of a hole formed in each of the first reservoirs 16a and 16b.

FIGS. 2(A) and 2(B) are views showing an electrophoretic member according to a second embodiment of the present invention. In the embodiment, channels 12a and 12b are formed in the plate member 10b as through-holes, and are connected to the both ends of the separating channel 12, respectively. Each of the first reservoirs 16a and 16b is formed of a cylindrical member having a bottom, and is provided with a through-hole having a diameter smaller than that of the first reservoirs 16a and 16b at a central portion of the bottom as the second reservoirs 14a and 14b. The first reservoirs 16a and 16b are attached to the plate member 10b, so that the second reservoirs 14a and 14b communicate with the channels 12a and 12b, respectively.

In the embodiments shown in FIGS. 1(A), 1(B), 2(A) and 2(B), it is preferable that at least the second reservoir 14a at a sample filling side (hereinafter, a suffix ‘a’ is designated as the sample filling side) has an inner wall with hydrophilicity, and the first reservoir 16a at the sample filling side has a bottom surface or an area covering from the bottom surface to an inner surface thereof with hydrophobicity.

FIG. 3 is an enlarged view showing a sample filling side of an electrophoretic member according to a third embodiment of the present invention, wherein the second reservoir 14a is formed on the bottom surface of the first reservoir 16a. In other words, on the bottom surface of the first reservoir 16a, a peripheral portion (shown as 14a) of a portion 12a communicating with the separating channel 12 is processed to be hydrophilic, and a portion outside thereof is processed to be hydrophobic. Accordingly, when a sample is injected, the sample is held at the portion processed to be hydrophilic. That is, the portion with hydrophilicity constitutes the second reservoir 14a. The hydrophilic area 14a has a size suitable for holding a sample having a quantity of several tens of nL to a few μL.

The surface process for imparting hydrophilicity and hydrophobicity includes various methods. For example, when a glass plate is used as the base plate, the glass plate is processed with acid to be hydrophilic, and is processed with a resin coating such as a fluoride resin, silane coupling agent and the like to be hydrophobic. Also, a hydrophilic material may be used as the base plate material 10b and a hydrophobic material may be used as the first reservoir 16a to provide hydrophilicity and hydrophobicity, respectively.

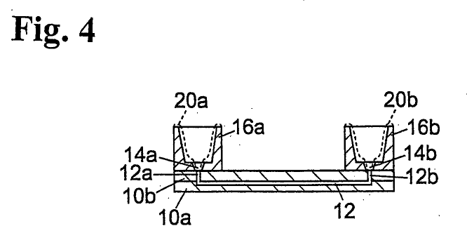

FIG. 4 is a sectional view showing an electrophoretic member according to a fourth embodiment of the present invention. In the embodiments shown in FIGS. 1(A) through 3, electrodes are provided independently from the first reservoirs 16a and 16b, and immersed in migration buffer in the first reservoirs 16a and 16b, respectively. In the embodiment shown in FIG. 4, electrodes 20a and 20b are integrally formed with the electrophoretic member beforehand, and extend from the first reservoirs 16a and 16b to the second reservoirs 14a and 14b.

The electrodes 20a and 20b are formed of a metal layer or a metal wire made of platinum. When the electrodes 20a and 20b are formed of a metal layer, the metal layer is formed with a depositing method or a sputtering method, and is then patterned with lithography and etching. In this case, it is preferable that the inner wall surfaces of the first reservoirs 16a and 16b are inclined to expand toward opening portions thereof as shown in FIG. 4, thereby making it easy to form the metal layer. Alternatively, a metal wire may be embedded in a resin and fixed to the first reservoirs 16a and 16b as the electrodes 20a and 20b. The electrodes 20a and 20b are exposed and contact the sample and the migration buffer in the second reservoirs 14a and 14b, and are exposed outside the first reservoirs 16a and 16b to be connected to lead wires of a power supply.

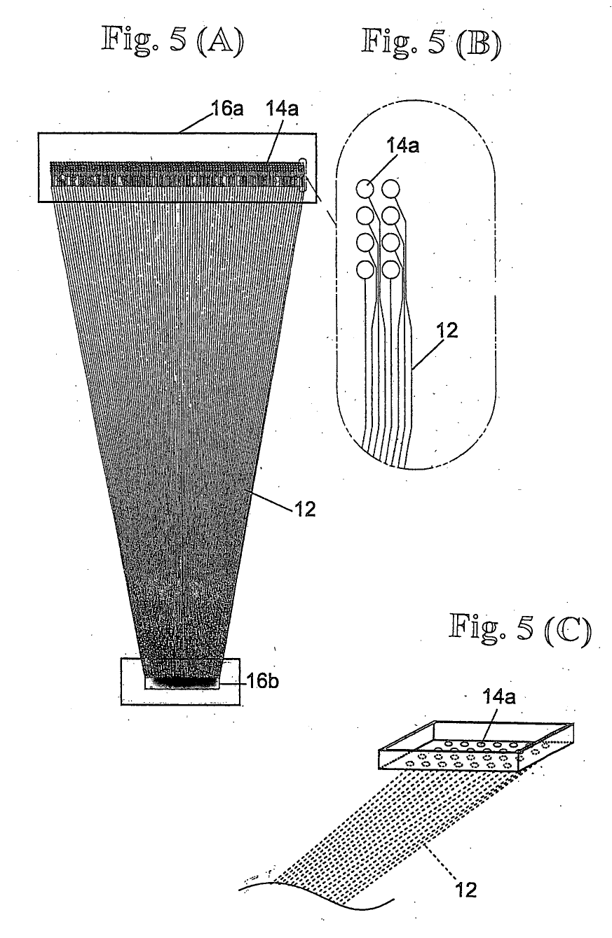

FIGS. 5(A) to 5(C) are views showing an electrophoretic member according to a still further embodiment of the present invention. In the embodiments shown in FIGS. 1(A) through 4, the separating channel 12 is formed in the base plate. In the embodiment shown in FIGS. 5(A) to 5(C), a plurality of separating channels 12 is formed in a common base plate. As shown in FIGS. 5(A) to 5(C), the separating channels 12 are arranged not to cross each other. Each of the separating channels 12 is provided with the second reservoir 14a at one end thereof for filling a sample, and has the other end connected to the common first reservoir 16b. The first reservoir 16a on one end side covers an entire area where the second reservoirs 14a are disposed to constitute a large common reservoir. As shown in FIG. 5(C), the second reservoirs 14a are provided in the common first reservoir 16a and connected thereto. The common first reservoir 16b on the other side also covers an entire area where the openings of the other sides of the separating channels 12 are disposed to thereby constitute a large common reservoir. The openings of the other end sides of the separating channels 12 are connected to the first reservoir 16b.

The electrodes may be provided to the first reservoirs 16a and 16b beforehand, respectively, or may be inserted separately. Also, the electrodes may be provided to each of the second reservoirs 14a on the sample filling side beforehand, or may be inserted separately.

A material for the plate members 10a and 10b constituting the base plate includes quartz glass, borosilicate glass, resin or the like. When a component separated through the migration is optically detected, a transparent material is selected. When detecting means other than light is used, the plate members 10a and 10b are not limited to a transparent material.

The separating channel 12 can be formed by means of lithography and etching (wet etching and dry etching). Also, the holes constituting the second reservoirs 14a and 14b or the channels 12a and 12b can be formed by means of a sand blast or laser drill.

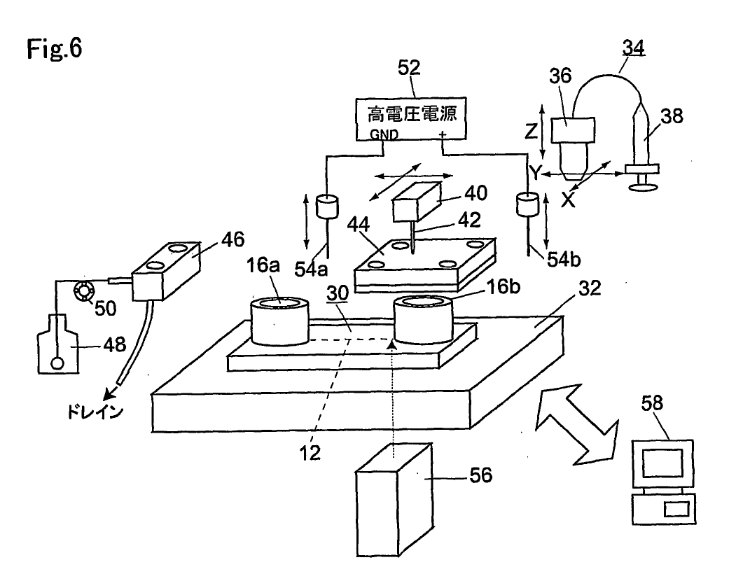

FIG. 6 is a schematic view showing an electrophoretic device using the electrophoretic member shown in FIGS. 1(A) through 3. An electrophoretic member 30 is disposed on a temperature regulating plate 32. A base plate of the electrophoretic member 30 as well as the temperature regulating plate 32 is made of a transparent material, and the temperature regulating plate 32 is provided with a heating device and a temperature regulating device to thereby maintain a constant temperature.

Reference numeral 34 represents a gel filling mechanism for filling a sieving medium to the electrophoretic member 30. The gel filling mechanism 34 includes a nozzle 36 for discharging the gel and a syringe 38 for sucking and discharging the gel. The nozzle 36 is supported to move horizontally on a flat plane in the X and Y directions and vertically in the Z direction, and fills the sieving medium to the first reservoir and the channel from the first reservoir 16a or 16b.

A dispending mechanism 40 is provided for injecting a sample to the second reservoir in the bottom portion of the first reservoir 16a at a cathode side. The dispensing mechanism 40 can move horizontally on a flat plane in the X and Y directions and vertically in the Z direction. A nozzle 42 of the dispensing mechanism 40 moves between a position of any well of a micro-titer plate 44 as a sample plate containing the sample and a position of the second reservoir in the first reservoir 16a, so that the sample in the well of the plate 44 can be injected into the second reservoir. Reference numeral 46 represents a port for cleaning the dispensing nozzle 42. A cleaning liquid 48 is supplied through a pump 50 and is discharged through a drain, so that the nozzle 42 is inserted into the cleaning port 46 for cleaning.

A high voltage power supply device 52 is provided for applying a voltage to introduce the sample into the separating channel from the second reservoir and separate the introduced sample electrophoretically. Electrodes 54a and 54b are connected to the power supply device 52. The electrode 54a is inserted into the migration buffer in the first reservoir 16a or the sample in the second reservoir, and the electrode 54b is inserted into the migration buffer in the first reservoir 16b to apply the voltage.

A multicolor fluorescent detecting system 56 is disposed below the temperature controlling plate 32 for detecting a sample component electrophoretically separated at a position on the end portion side of the separating channel 12 in the electrophoretic member 30 opposite to the second reservoir for filling the sample. A light source of the detecting system 56 irradiates exciting light such as argon laser on the separating channel 12, and the optical system 56 detects fluorescence generated through excitation of exciting light. When a sample such as DNA is analyzed, DNA segments are labeled with four types of fluorescent dyes according to an end base, and are electrophoretically separated. Each of the fluorescent dyes is detected by one of four fluorescent wavelengths.

Reference numeral 58 represents a personal computer (PC) as a control/data processing unit for processing data based on a fluorescent signal received by the detecting system 56 and for controlling the electrophoretic device. The personal computer 58 controls various operations of various portions such as filling the sieving medium, filling the sample, and applying the voltage to the electrophoretic member 30, so that the optical system 56 obtains the fluorescent detecting signal, and the migrating pattern is formed based on the obtained fluorescent signal, thereby determining a base sequence.

The separated components through the migration are detected with a fluorescence intensity method, and may be detected with other detecting methods such as an absorption photometry, an electrochemical method, and an electrical conductivity method.

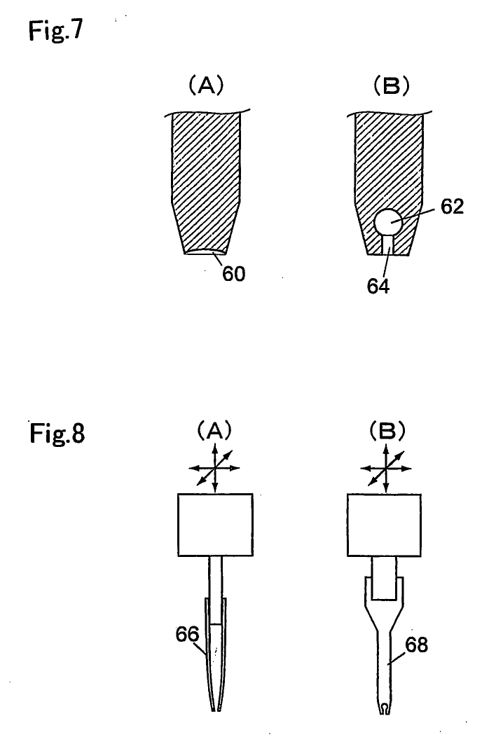

A dispensing nozzle 42 of the sample dispensing mechanism 40 has a leading end formed in a shape shown in FIG. 7(A) or 7(B), so that a minute amount of the sample can be dispensed. The nozzle shown in FIG. 7(A) includes a depressed portion 60 at the leading end thereof. The depressed portion 60 has a diameter of 10 μm to 5 mm, preferably 2 mm, and a depth of 5 μm to 1 mm, preferably 200 μm, so that a sample of several tens of nL to a few μL can be held in the depressed portion 60 through surface tension and transferred to the second reservoir in the first reservoir 16a to be dispensed.

The nozzle shown in FIG. 7(B) includes a cavity 62 at a leading end thereof. The cavity 62 has an open leading edge with a groove 64 extending to the leading edge. The groove 64 and the cavity 62 have sizes for retaining a sample of several tens nL to a few μL in a region from the groove 64 to the cavity 62. The sample is received in the region from the groove 64 to the cavity 62 through capillary phenomenon, and is transferred to the second reservoir in the first reservoir 16a to be dispensed.

FIGS. 8(A) and 8(B) are schematic views showing nozzles of the sample dispensing mechanism 40 together with transmitting mechanisms. As shown in FIG. 8(A), the nozzle includes a dispensing nozzle tip 66 at a leading end thereof, wherein a sample is sucked into the tip 66 through capillary phenomenon, and is transferred to the second reservoir in the first reservoir 16a to be dispensed. As shown in FIG. 8(B), the nozzle includes the nozzle 68 shown in FIG. 7(B) at a leading end thereof, and is used as a probe for dispensing a minute quantity of the sample.

Next, results of the electrophoretic separation according to the embodiment will be explained in comparison with a conventional cross injection method.

### PREPARATION EXAMPLE 1

### DNA Sample, Sieving Medium and Buffer

DNA sample, sieving medium and buffer as follows were used in Examples 1 and 2, and Comparative Example 1 described below.

Cast sample: pUC18 plasmid DNA

Sanger reagent: BigDye™ Ver.3.1 (product of Applied Biosystems)

Purification: Ethanol precipitation method

Sample loading buffer: Milli-Q water or 50-80% formamide

Sieving medium: 2% linear polyacrylamide gel (x1TTE), 7M urea

Migration buffer: x1 Tris-TAPS-EDTA (TTE)

### EXAMPLE 1

### Electrophoretic Migration Based on the Present Invention Using the Electrophoretic Member in Which the Single Separation Channel is Formed in the Single Base Plate

The electrophoretic member shown in FIG. 1 in which the single separation channel is formed in the single base plate was used. A process of the electrophoretic migration is as follows:

1) The sieving medium was filled by a syringe under an increased pressure from the anode side of the separating channel.

2) The migration buffer was filled in the respective first reservoirs, and the electrodes were immersed into the respective first reservoirs. A high voltage (125 V/cm, for 5 minutes) was applied to the electrodes to perform a pre-run for removing foreign ions in the gel.

3) After the migration buffer in the first reservoir on the cathode side was sucked, 3 μL of a DNA sample was injected into the second reservoir.

4) The electrodes were immersed into the respective second reservoirs and a voltage (50 V/cm, for 40 seconds) was applied thereto in order to introduce the sample.

5) After introduction of the sample, the application of the voltage was stopped, and the migration buffer was filled into the first reservoir on the cathode side. At this time, even if the sample remained in the second reservoir, it was diluted by the migration buffer.

6) A high voltage was applied for the migrating separation. It was preferred that the voltage applied at that time was 70-300 V/cm and, as an example, 125 V/cm was used.

7) Segments of the DNA sample electrophoretically separated at the detecting section were detected optically or electrochemically in chronological order, and data was processed to thereby obtain an electropherogram.

### COMPARATIVE EXAMPLE

### Electrophoretic Migration Based on a Conventional Cross Injecting Method

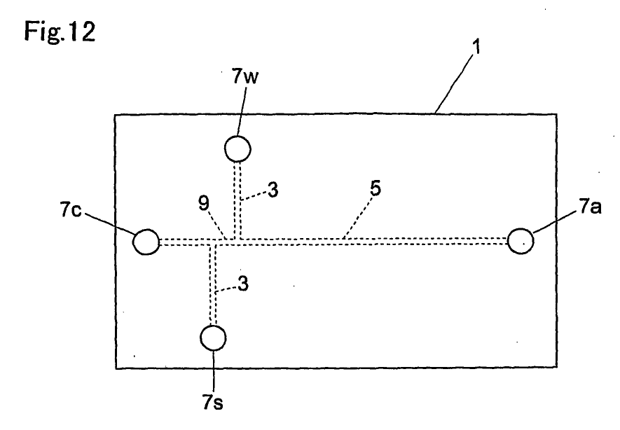

The electrophoretic member shown in FIG. 12 was used. A process of the electrophoretic migration is as follows:

1) The sieving medium was equally and uniformly filled into three-way branched channels from a cross section on the anode side of the separating channel by a syringe under an increased pressure.

2) The migration buffer was filled in the respective wells, and the electrodes were immersed into the wells. A high voltage (125 V/cm) was applied to the electrodes for 5 minutes to perform pre-run and pre-loading.

3) After the migration buffer in the sample well was sucked, 8 μL of a DNA sample was injected.

4) The electrodes were immersed in a sample reservoir and a sample waste reservoir, and the voltage was applied between the loading channels. The applied voltage and time at this time were 125 V/cm and 10 minutes.

5) After the sample was introduced, the application of the voltage was once stopped, and a high voltage was applied between the anode and cathode. It was preferred that the voltage applied at this time was 70-300 V/cm, and, in the present experiment, 125 V/cm was employed.

6) In parallel with the step of 5), a voltage for pulling back was applied to the sample reservoir and the sample waste reservoir for 200 seconds. It was preferred that the voltage applied at this time was 10-100 V/cm, and 60 V/cm was applied in this experiment. After the application of the voltage for 200 seconds, the application of the voltage was stopped.

7) Segments of the DNA sample electrophoretically separated at the detecting section were detected optically or electrochemically in chronological order, and data was processed to thereby obtain an electropherogram.

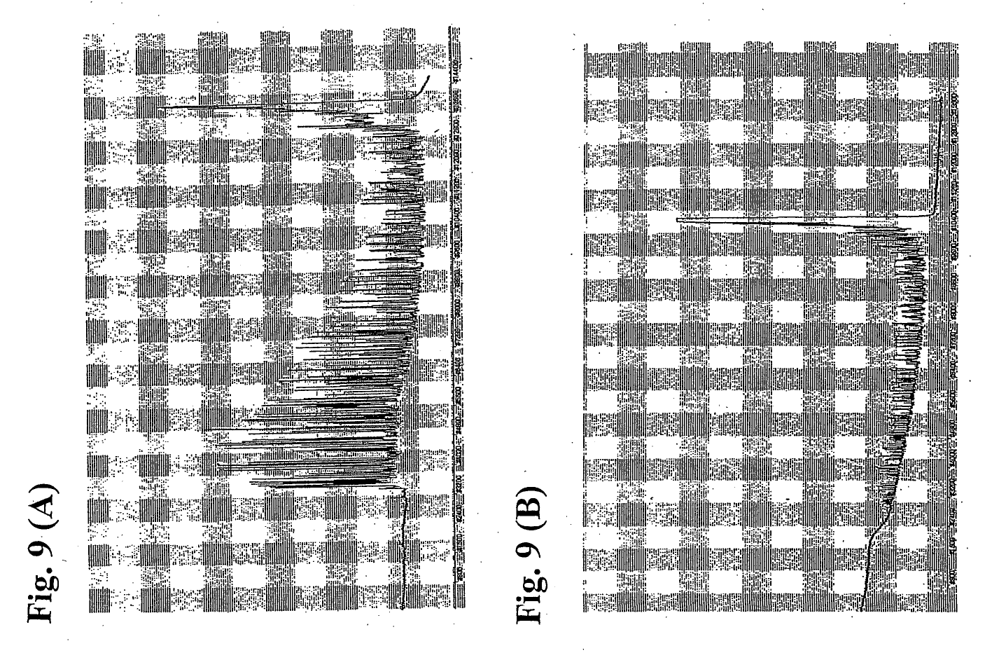

Examples of the migrating patterns obtained as described above are shown in FIGS. 9(A) and 9(B). FIG. 9(A) shows the result of Example 1; and FIG. 9(B) shows the result of Comparative Example. These migrating patterns were obtained by irradiating exciting light to the DNA sample electrophoretically separated at the detecting portion to detect the fluorescence. The abscissa axis represents a scanning number corresponding to time when exciting light was scanned. The ordinate represents fluorescent intensity. When FIGS. 9(A) and 9(B) are compared, according to the embodiment of the invention, high signal levels of the fluorescence detection at the respective peaks electrophoretically separated are obtained with good resolution.

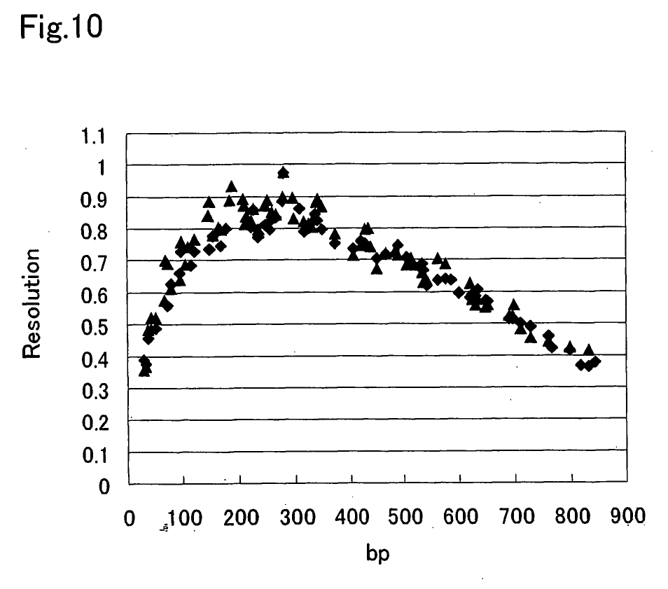

FIG. 10 shows results wherein resolutions of the migrating patterns are compared between Example 1 of the present invention and Comparative Example. A diamond represents data of the present embodiment, and a triangle represents data of the comparative example. The abscissa axis represents base pair (bp), and the ordinate represents resolution. When resolution is larger than 0.5, two peaks are separated, and when resolution is less than 0.5, the two peaks are overlapped to form one peak. Regarding the resolution, the embodiment and the comparative example have almost the same patterns, and there is practically no difference. Also, there is no influence of the remaining sample without an additional sample washing step.

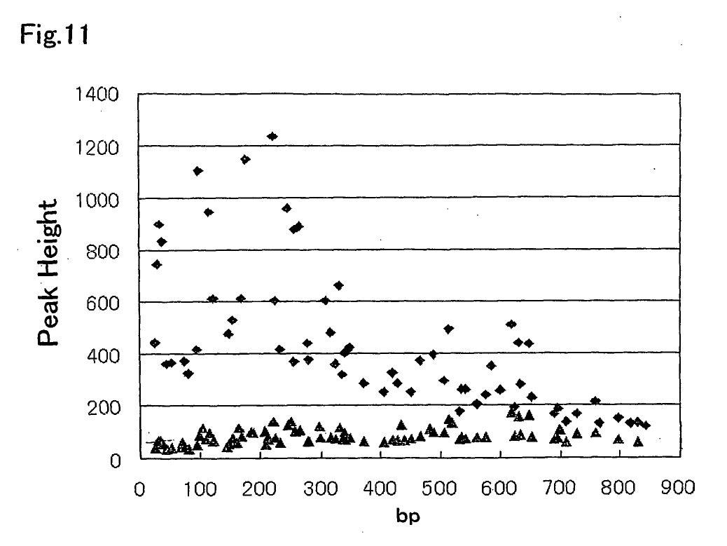

FIG. 11 shows comparison of signal levels represented in peak heights. As apparent also from FIGS. 9(A) and 9(B), the present embodiment obtains higher signal levels as compared with the comparative example.

### EXAMPLE 2

### Electrophoretic Migration Based on the Present Invention Using the Electrophoretic Member in which a Plurality of the Single Separation Channels is Formed in the Common Base Plate

The electrophoretic member shown in FIG. 5 in which the separation channels (384 channels) are formed in the common base plate was used. A process of the electrophoretic migration was the same as that of Example 1.

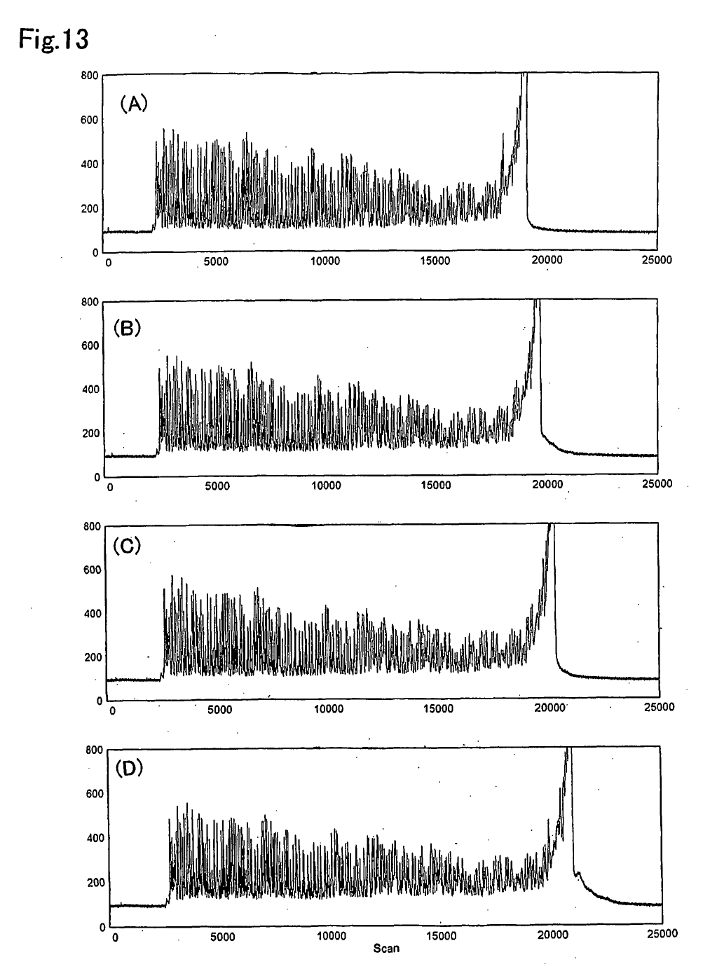

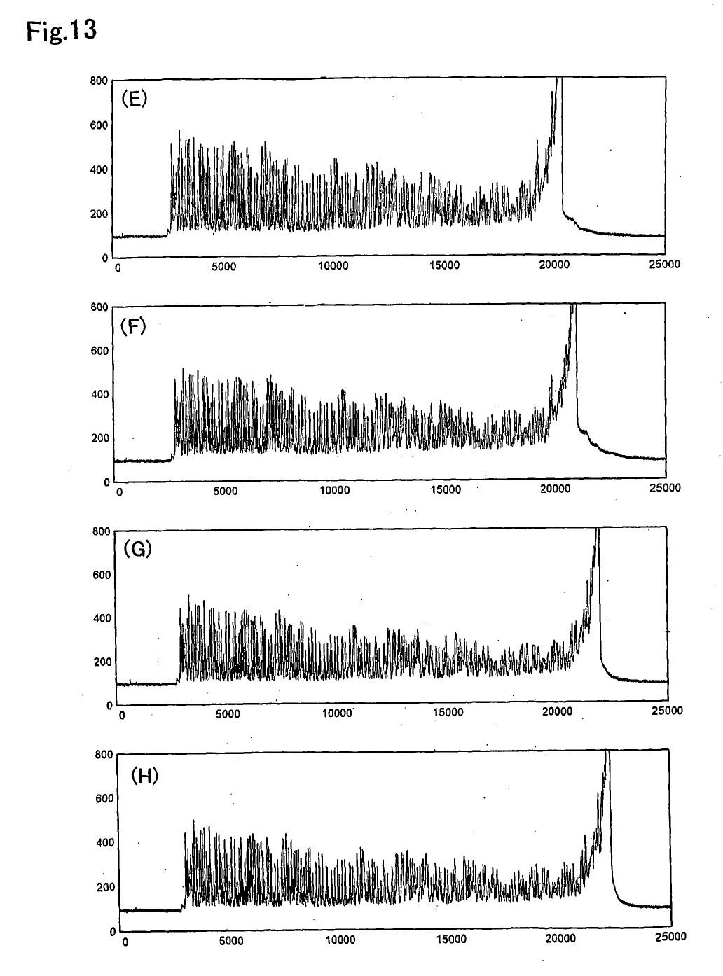

FIGS. 13(A) to 13(H) are graphs showing migration patterns of the 129th to 135th separation channels from the left side as examples among the 384 separation channels of the electrophoretic member. As shown in FIGS. 13(A) to 13(H), each of the separation channels exhibits good separation with a stable base line. Also, the electrophoretic migration shows good reproducibility among the separation channels. Large peaks shown in the separation channels (portions showing all sample fragments as one large peak with no separation) appear in adjacent channels, indicating that there is no cross-talk of the sample among the separation channels. That is, even if the electrophoretic member has a plurality of the separation channels with the common first reservoir at the sample injection side, since the sample injected in the second reservoir is diluted by the migration buffer, there is no influence on the migrating pattern in each of the separation channel.

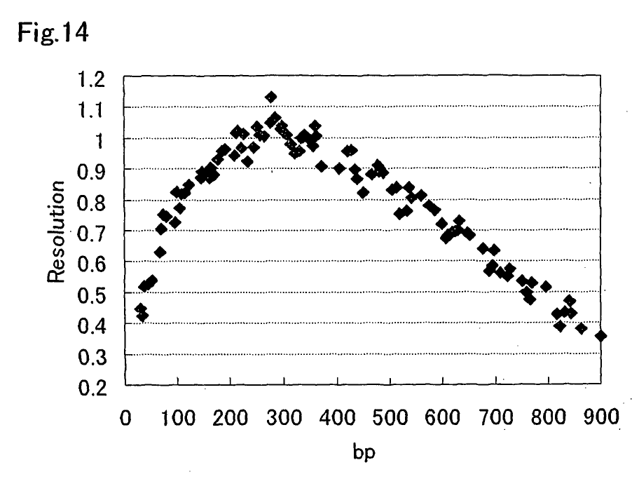

FIG. 14 is a graph showing resolution of typical one of the 384 separation channels (129th from the left side). As compared with the resolution of Example 1 shown in FIG. 10, good separation was obtained for the separation channels shown in FIG. 13(A) to 13(H).

According to the invention, the electrophoretic member, the electrophoretic device using the same, the electrophoretic method, and the sample dispensing probe are applicable for electrophoretically separating a minute quantity of protein, nucleic acid, chemicals and the like at a high speed with high resolution in the fields of biochemistry, molecular biology and clinic.

The disclosure of Japanese Patent application No. 2003-330614, filed on Sep. 22, 2003, is incorporated in the application.

While the invention has been explained with reference to the specific embodiments of the invention, the explanation is illustrative and the invention is limited only by the appended claims.

1. An electrophoretic member comprising:
a base plate,
at least one separating channel disposed in the base plate for electrophoretically separating a sample along the channel by applying a voltage between two end portions thereof, said separating channel being formed in a single channel,
first reservoirs disposed at the two end portions of the separating channel and communicating with the separating channel for reserving liquid, and
at least one second reservoir provided inside at least one of the first reservoirs disposed on the two end portions of the separating channel, said at least one second reservoir being located at an end of a sample injecting side of the first reservoirs for injecting the sample.

2. An electrophoretic member as claimed in claim 1, wherein said at least one second reservoir is formed in a depression having a diameter smaller than that of the first reservoirs.

3. An electrophoretic member as claimed in claim 2, wherein said at least one second reservoir has an inner wall with hydrophilicity and one of said first reservoirs has at least a bottom surface with hydrophobicity.

4. An electrophoretic member as claimed in claim 1, wherein said one of the first reservoirs has a portion communicating with the separating channel at a bottom surface thereof, said portion having a peripheral portion surface-processed to be hydrophilic, and an outer side of the peripheral portion to be hydrophobic so that the second reservoir is formed at the bottom surface of the one of the first reservoirs.

5. An electrophoretic member as claimed in claim 1, further comprising integrated electrodes provided in the first and second reservoirs, respectively.

6. An electrophoretic member as claimed in claim 1, wherein said separating channel is plural and at least one said first reservoir is common to the separating channels.

7. An electrophoretic device comprising said electrophoretic member as claimed in claim 1, a power supply device for applying a migration voltage between the two end portions of the separating channel, a sample dispensing probe for dispensing a minute amount of the sample into the second reservoir, and a detecting device disposed at an end portion of the separating channel opposite to the sample injecting side for detecting a sample component migrating along the separating channel.

8. An electrophoretic device as claimed in claim 7, wherein said sample dispensing probe includes at a leading end thereof means for taking and dispensing the minute amount of the sample through at least one of capillary phenomenon and surface tension.

9. An electrophoretic device as claimed in claim 8, wherein said taking and dispensing means is one of a depression and a groove.

10. An electrophoretic separation method using the electrophoretic member as claimed in claim 1, comprising:
filling a sieving medium into the at least one separating channel of the electrophoretic member,
electrophoretically introducing the sample into the channel by injecting the sample from one of the first reservoirs at the sample injection side to the at least one second reservoir, and filling a migration buffer into the other of the first reservoirs,
filling the migration buffer into the first reservoir at the sample injecting side while leaving the sample in the second reservoir after the sample is introduced, and
carrying out migrating separation by applying the voltage between the two end portions of the separating channel.

11. A sample dispensing probe having at a leading end thereof means for taking and dispensing a minute amount of a sample through at least one of capillary phenomenon and surface tension.

12. A sample dispensing probe as claimed in claim 11, wherein said means is one of a depression and a groove.
